(19) United States
(12) Patent Application Publication
(10) Pub. No.: US 2009/0246171 A1
(43) Pub. Date: Oct. 1, 2009

(54) AUTOMATIC SYSTEM FOR DOSE CONTROL IN TREATING HEPATITIS C USING INFUSION PUMPS

(76) Inventors: William P. Van Antwerp, Valencia, CA (US); Rebecca M. Bergman, North Oaks, MN (US); Rachael M. Scherer, Plymouth, MN (US)

Correspondence Address: FOLEY & LARDNER, 555 South Flower Street, SUITE 3500, LOS ANGELES, CA, 90071-2411, US

(21) Appl. No.: 12/257,307

(22) Filed: Oct. 23, 2008

Related U.S. Application Data
(60) Provisional application No. 61/040,026, filed on Mar. 27, 2008.
(60) Provisional application No. 61/058,001, filed on Jun. 2, 2008.
(60) Provisional application No. 61/040,038, filed on Mar. 27, 2008.
(60) Provisional application No. 61/058,006, filed on Jun. 2, 2008.
(60) Provisional application No. 61/040,014, filed on Mar. 27, 2008.
(60) Provisional application No. 61/040,029, filed on Mar. 27, 2008.
(60) Provisional application No. 61/082,672, filed on Jul. 22, 2008.

(51) Int. Cl. A61K 38/21; A61P 31/12; G06Q 50/00

(52) U.S. Cl. 424/85.4; 705/2

(57) ABSTRACT

According to one embodiment, a method of controlling a plurality of treatment dosages for Hepatitis C includes: setting a first dosage level to cause a viral load of a subject user to reduce at a rate over time; determining, after setting the first dosage level, if the rate of the viral load reduction changes over time by a first threshold; setting a second dosage level in response to determining that the rate of the viral load reduction changes by the first threshold; determining, after setting the second dosage level, if the rate of the viral load reduction changes over time by a second threshold; setting a third dosage level in response to determining that the rate of the viral load reduction changes by the second threshold; and maintaining the second dosage level in response to determining that the rate of the viral load reduction has not changed by the second threshold.

### CROSS-REFERENCE TO RELATED PATENT APPLICATIONS

The following applications were filed by the present Applicant and are incorporated herein by reference in their entireties: U.S. Provisional Patent Application No. 60/997,897, filed Oct. 5, 2007; U.S. Provisional Patent Application No. 61/040,026, filed Mar. 27, 2008; U.S. Provisional Patent Application No. 61/058,001, filed Jun. 2, 2008; U.S. Provisional Patent Application No. 61/040,038, filed Mar. 27, 2008; U.S. Provisional Patent Application No. 61/058,006, filed Jun. 2, 2008; U.S. Provisional Patent Application No. 61/040,014, filed Mar. 27, 2008; U.S. Provisional Patent Application No. 61/040,029, filed Mar. 27, 2008; and U.S. Provisional Patent Application No. 61/082,672, filed Jul. 22, 2008.

### BACKGROUND OF THE INVENTION

The present invention relates generally to the field of autonomous delivery of infusion media and, in particular, to the delivery of infusion media for the treatment of Hepatitis C virus (HCV) infection.

Chronic HCV infection is one of the most common chronic infections in the United States and many industrialized countries. In the United States, more than 4 million people have been infected with HCV.

The drug interferon has been used in the treatment of HCV infection. When used in (or part of) a systemic therapy, forms of interferon, such as interferon-alpha and interferon-gamma, are administered to patients—typically by intramuscular injection. The injection of interferon in a muscle, in a vein, or under the skin is generally well tolerated.

An example of a current standard of care involves supplying HCV-infected patients with weekly dosages of pegylated interferon (and, possibly, concomitant weight-based oral ribavirin for controlling the patient's red blood cell count). The weekly dosages of interferon are typically supplied over the length of the treatment period and may be generally equal to each other in size (milliliters). For example, a dosage of a generally static size may be supplied to the patient during each week of the treatment period. (However, newer treatment modalities and therapies for HCV involving a continuous delivery of interferon have been described in more detail, for example, in the related applications identified above.)

Under the example of the current standard of care described above, cure rates can be only about 50% in controlled clinical trial settings. Outside of such settings, cure rates can be considerably lower in environments such as community practices, due in part to inefficient therapy monitoring and tracking systems.

### SUMMARY OF THE DISCLOSURE

Embodiments of the invention relate to medical data management systems and processes for managing data relating to one or more medical or biological conditions (such as, but not limited to, Hepatitis C virus (HCV) infections) of at least one (or a plurality of) subject(s) over a wide area network, such as the Internet, and which seek to achieve cure rates higher than those noted above.

Embodiments of such systems and processes provide various functions for subject-users, healthcare provider-user, payor-users, pharmacy-users and combinations thereof, for improved treatment for HCV and medical data management for individual subjects and/or groups of subjects. For example, embodiments of the system allow collection and analysis of aggregate data from many HCV subject sources, for improving overall healthcare practices for individual patents and/or groups of subjects.

### BRIEF DESCRIPTION OF THE DRAWINGS

FIG. 1 is a generalized schematic diagram of an HCV system environment according to an embodiment of the present invention.

FIG. 2 shows a graph of a typical HCV patient response to treatment and a graph of a controlled treatment according to an embodiment of the present invention.

FIG. 3 shows a graph of a typical HCV patient where the response to treatment is satisfactory and a typical unsatisfactory HCV patient response to treatment.

FIG. 4 is a generalized flowchart showing a process for controlling HCV treatment dosages according to an embodiment of the present invention.

FIG. 5 is a graph showing two different patient responses to HCV treatment.

FIG. 6 is a graph showing three different patient responses to HCV treatment.

FIG. 7 is a graph showing two different patient responses to HCV treatment.

### DETAILED DESCRIPTION

Embodiments of the invention relate to medical data management systems and processes for managing data relating to treating medical conditions (such as, but not limited to, Hepatitis C virus (HCV) infections). According to particular embodiments, treatment is managed and controlled (e.g., over a wide area network, such as the Internet) to successfully treat afflicted subject users.

According to embodiments where HCV infections are treated, it is contemplated that the infections may involve one or more HCV genotypes. HCV) is a positively stranded RNA virus that exists in at least six genetically distinct genotypes. These genotypes are designated Type 1, 2, 3, 4, 5 and 6, and their full length genomes have been reported (see, e.g., Genbank/EMBL accession numbers Type 1a: M62321, AF009606, AF011753, Type 1b: AF054250, D13558, L38318, U45476, D85516; Type 2b: D10988; Type 2c: D50409; Type 3a: AF046866; Type 3b: D49374; Type 4: WC-G6, WC-G11, WG29 (Li-Zhe Xu et al, J. Gen. Virol. 1994, 75: 2393-98), EG-21, EG-29, EG-33 (Simmonds et al, J. Gen. Virol. 1994, 74: 661-668), the contents of which are incorporated by reference in their entireties). In addition, viruses in each genotype exist as differing “quasispecies” that exhibit minor genetic differences. The vast majority of infected individuals are infected with genotype 1, 2 or 3 HCV. As such, this disclosure contemplates the treatment of one or more genotypes including, but not limited to, HCV genotype 1, HCV genotype 2 and/or HCV genotype 3.

Embodiments of such systems and processes provide various functions that may involve the participation of subject users, healthcare provider users, payor users, pharmacy users and combinations thereof, for improving treatment success statistics.

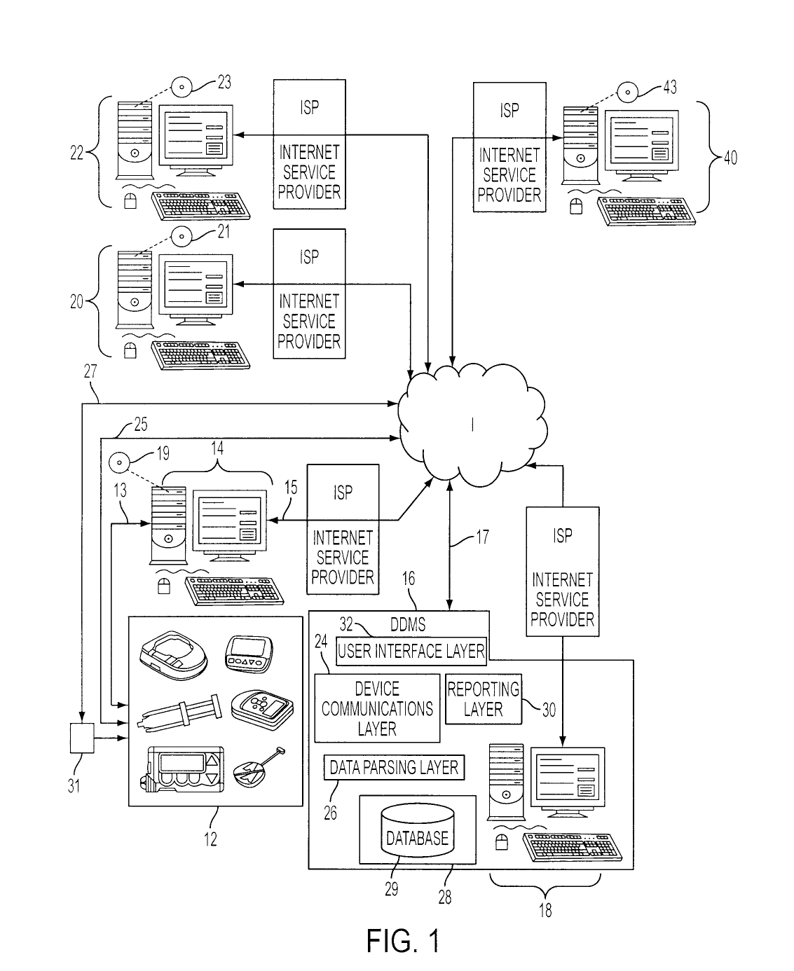

A generalized diagram of a system 10 according to embodiments of the present invention is shown in FIG. 1. Example embodiments of the system 10 are described herein with reference to usage in medical service contexts. In such embodiments, one or more (preferably multiple) subjects are each provided with at least one (or multiple) subject support device(s) 12.

A subject support device 12 may include a device that is designed to be carried by a subject or otherwise be in the subject's locality and provide a function, such as, but not limited to a treatment, metering, monitoring or sensing function on the subject or subject's environment. In certain embodiments, the subject support device may be or include a meter sensor (such as a biological sensor) that continuously or intermittently senses a condition or collects data regarding a condition over time. A subject support device 12 may include, for example, but without limitation, an infusion pump or other infusion device for dispensing a controlled amount of an infusion medium (such as, but not limited to, interferon) to a subject, a meter for monitoring viral loads or body temperature or other biological or medical condition over time, implantable or external sensors or meters for sensing or monitoring viral loads, infectivity, hemodynamic pressure or other biological conditions or medical events, including but not limited to cardiac events that can be monitored with a cardiac pacemaker, other electronic cardiac treatment device(s), or the like.

Representative and non-limiting examples of medical devices that may be employed as a subject support device according to certain embodiments of the invention are described in U.S. Pat. No. 6,641,533, titled “Handheld Personal Data Assistant (PDA) With A Medical Device And Method Of Using The Same” and U.S. patent application Ser. No. 10/033,724, titled “Infusion Device And Driving Mechanism For Same” (each of which is assigned to Medtronic MiniMed, Inc., and each of which is incorporated herein by reference in its entirety). Other suitable subject support devices may include, but are not limited to, medical infusion pumps manufactured by Medtronic MiniMed and distributed under the model names Paradigm™ 512/712, Paradigm™ 511 and MiniMed 508. Representative examples of meter devices that may be employed as a subject support device include, but are not limited to, meters manufactured or otherwise provided by Medtronic MiniMed (including Paradigm Link™ and BD Logic™), by Ascensia/Bayer (including DEX™-DEX2™ and Elite™-EliteXL™), or by LifeScan (including OneTouch™ Profile™, OneTouch™ Ultra™, OneTouch™ Basic™, Fast Take™, and SureSte™).

In further embodiments, a subject support device, such as an infusion pump as described above, may include an interface for communicating with one or more meters or sensors as described above and a memory for storing sensor or meter information. The interface may include an electrical port on each of the infusion pump and the sensor or meter through a hard-wire connector. Alternatively, the interface may include a wireless interface, such as an RF link, an optical link, a magnetic link or other suitable communication links. In this manner, an infusion pump may store information received from a sensor or meter over a period of time and, upon coupling the infusion pump to the network (as described herein), the stored information may be communicated to a system 16 server. Thus, the infusion pump may facilitate the collection and storage of information from one or more sensors and/or meters, and may communicate such information to the system 16 (along with stored pump setting or pump operation information) at a suitable time during a network communication session.

As will be described in more detail below with reference to certain embodiments, the information may be provided by a medical testing facility (or clinical laboratory or the like) in communication with the system 16. For example, the patient may make periodic (e.g., weekly) visits to the facility, where one or more pieces of his biological information are measured. As will also be described in more detail below, in further embodiments, the information may be measured in settings other than medical facilities or hospitals.

In the system 10 of FIG. 1, subject support devices 12 of one or more subjects may be connected in communication with respective subject-side computers 14. Each subject may have one or more subject support device 12 and at least one subject or subject-side computer 14. Each subject-side computer may be connectable to a wide area network, such as the Internet. The system 10 also includes a medical data management system 16 connected to the wide area network and which is described in more detail below.

Depending upon the environment of use, embodiments may also include additional network devices, such as additional computers, connected in the system 10 through the wide area network. For example, as part of the data management system 16 (or as a separate element), one or more system personnel, such as customer service operators and/or system administrators, may be connected for communication in system 10, via a computer or other suitable network device 18. Such system personnel may be trusted individuals, employed by (or otherwise associated with) an entity administering the system 16, such that appropriate security and controls may be implemented for system personnel handling or having access to subject information. In some embodiments described below, system personnel may include physicians, pharmacists or other trained medical personnel who may have access to some or all subject information stored on the system 16, to provide assistance to subject-users and/or healthcare provider-users.

Alternatively or in addition, one or more service providers may be connected to the network for communication in the system 10, each via a respective computer or suitable network device 20. In the context of a health care system, a service provider may be, for example, but without limitation, a healthcare provider such as a doctor, or authorized personnel at a doctor's office, a hospital, a laboratory, a treatment center or the like. One or more payor entities also may be connected to the network for communication in the system 10, each via a respective computer or suitable network device 22. In the context of a health care system, the payor entity may be, for example, an insurance company, or the like. One or more distribution entities may also be connected to the network for communication in the system 10, each via a respective computer or suitable network device 40. In the context of a health care system, the distribution entity may be, for example, a pharmacy, or the like.

The computers or other network devices 14, 18, 20, 22 and 40 may each include a conventional personal computer or other suitable network-connectable communication device having data processing capabilities. For some embodiments with limited functions, the network device may include, for example, but without limitation, a personal digital assistant (PDA), a mobile telephone, a pager, a dedicated medical communication device, or the like. Depending upon the embodiment and environment of use, the computers or other network devices 14, 18, 20, 22 and 40 may include or otherwise be associated with a user input device (such as, but not limited to, a keyboard, mouse, touch screen, optical input device, or the like) and a display device (such as, but not limited to, a cathode-ray tube monitor, an LCD display, an LED display, a plasma display or the like). For convenience and simplification of the present disclosure (and without limiting the present invention), embodiments are described herein with reference to the network devices 14, 18, 20, 22 and 40 as computers.

The communication link 13 between each subject support device 12 and a subject-side computer 14 may be provided in any suitable manner including, but not limited to a direct or indirect hard wired connection (for example, through conventional communication ports on the device 12 and computer 14, such as a serial port, parallel port, RS-232 port, USB port or the like), a wireless connection (for example, a radio-frequency or other magnetic or electromagnetic link), an optical connection, a combination of the forgoing, or the like. For embodiments employing wireless or optical connections, the subject support device 12 and the subject-side computer 14, each include suitable wireless and/or optical transmitters and receivers for communication therebetween. In yet further embodiments, the subject support device 12 may be configured with suitable hardware and software to enable a direct connection of the subject support device 12 to the network (such as the Internet, LAN or extranet), as shown in FIG. 1, at reference number 25. Alternatively, or in addition, as shown at reference number 27 in FIG. 1, the subject support device may be connectable to the network (such as the Internet, LAN or extranet) through a separate network connection device 31 that provides some or all of the hardware and/or software for connection of the subject support device 12 to the network and communication on the network.

In one example embodiment, the communication link 13 employs a communication link device that interfaces with the subject support device 12 and connects to the subject-side computer 14 through a connector cable or the like. Example communication link devices include, but are not limited to, Com-Station™, ComLink™ or Paradigm Link™ devices. The connector cable may include, but is not limited to, a serial cable connector a BD-USB connector, or the like.

In a further embodiment, the communication link device may include a communication cradle (not shown) having a receptacle in which the subject support device 12 is configured to be set or installed for communication with the subject-side computer 14. The subject support device 12 may include electrical contacts, magnetic and/or optical connections that engage corresponding contacts or connections in or on the cradle, such that, upon setting the subject support device 12 in the cradle, a communication connection is made between the device 12 and the cradle. The cradle may, in turn, be connected by a wired or a wireless communication link to the subject-side computer 14.

The cradle allows a user to quickly and easily connect a subject support device to a subject-side computer 14, thus simplifying various activities and functions described herein. Thus, by setting the subject support device 12 in or on the cradle, an electronic communication link is created between the subject support device 12 and the subject-side computer 14. Examples of a communication cradle and other suitable communication links 13 are described and shown in published PCT Application No. PCT/US99/22993, titled “Communication Station And software For Interfacing With An Infusion Pump, Analyte Monitor, Analyte Meter, Or The Like” (which is incorporated herein by reference in its entirety).

With the communication link 13 coupling a subject-side computer 14 to one or more subject support devices 12, information including data, programs, updated software or the like may be transferred between the computer 14 and the device(s) 12. As described above, each subject-side computer 14 is also coupled for communication over a wide area network, such as the Internet, through a respective second communication link 15. The second communication link 15 may include any suitable communications connection and may employ, for example, a suitable Internet Service Provider (ISP) connection to the Internet and/or include a hard wired connection, a wireless connection, an optical connection, a combination of the forgoing, or the like. While not shown in the drawing, suitable modem, cable-modem, satellite, DSL or other system elements may be employed for connecting the subject-side computer 14 to the Internet. Similar communication links may be employed for connecting computers 18, 20, 22 and 40 for communication over the Internet.

The medical data management system 16 is coupled for communication over the wide area network, such as the Internet, through one or more further communication links 17. The link(s) 17 may include any suitable communications connection and, for example, may employ one or more suitable Internet Service Provider (ISP) connections to the Internet and/or a hard wired connection, a wireless connection, an optical connection, a combination of the forgoing, or the like. While not shown in the drawing, suitable modem, cable-modem, satellite, DSL or other system elements for connecting the medical data management system 16 to the wide area network may be employed.

The medical data management system 16 includes software that runs on at least one (or multiple) server(s) connected to the Internet. The system 10 may also include additional system software 19 residing on the subject-side computer 14, software 21 residing-on the healthcare provider's computer 20, software 23 residing on the payor entity computer 22, and software 43 residing on the pharmacy entity computer 40 for interacting with the medical data management system 16 and providing functions described herein. The software 19, 21, 23 and 43 may be stored in a hard-disc or other suitable computer readable storage device connected to the respective user computers 14, 20, 22 or 40. The software 19, 21, 23 and 43 may be supplied to the respective users by any suitable means, including, but not limited to computer readable discs delivered to the user by mail or other form of delivery, or by uploading such software to the user computers 14, 20, 22 or 40 from the system 16, through an Internet connection, for example, during a suitable registration procedure.

Other system software (not shown) may be provided on the operator or administrator computer(s) 18, for providing similar functions and/or other functions for which the operator or administrator may be authorized to perform. The software for system 16 and the software residing on computers 14, 20, 22 and 40 may be configured using any suitable standard or non-standard software coding techniques to provide functions described herein. Alternatively, or in addition, the functions of the management system 16 and/or the user computers 14, 20, 22 and 40 described herein may be implemented in suitably configured hardware circuitry or combinations of hardware and software.

In general, the medical data management system 16 may be configured to provide any one or combination of functions to provide an expanded capability to treat individual subjects, as well as groups of subjects with similar medical conditions or other characteristics. In particular embodiments, the system 16 may be configured to treat such individuals for HCV infection.

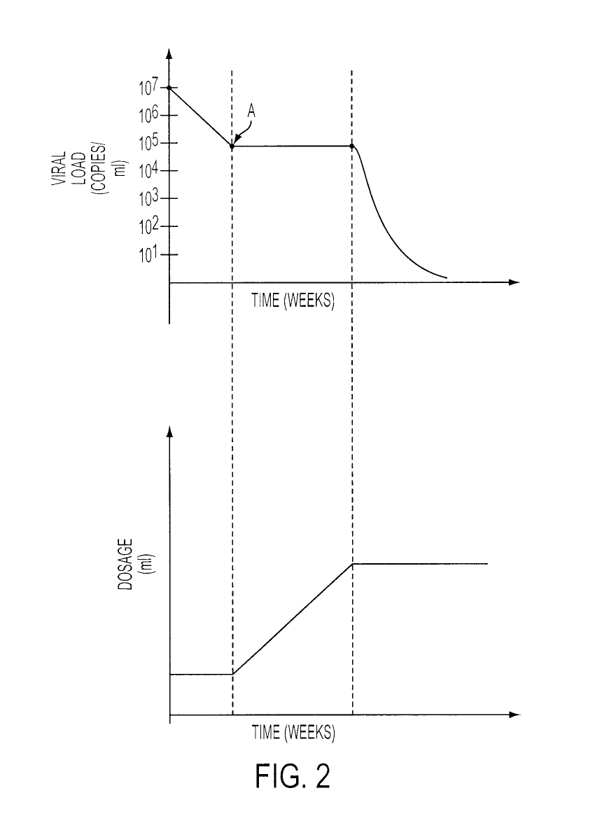

With reference to the upper graph of FIG. 2, the kinetics (i.e., viral load in the bloodstream) of a typical (or hypothetical) patient is shown. While the curve may differ to some extent for each different patient, the viral load of the hypothetical patient, shown in FIG. 2, generally decreases over time (e.g., a period of approximately 48 weeks) in response to the application of interferon during that time.

As shown in the upper graph of FIG. 2, the response includes three separate phases. During an initial phase (occurring, for example, over the first 1-2 weeks of treatment), the slope of the response is negative or downward and relatively steep. As shown in the response, the viral load decreases by a factor of around 100, from about around 1×10⁷ copies/milliliter (ml) to around 1×10⁵ copies/ml. This relatively rapid decrease may reflect the rather immediate reduction in HCV in the patient's bloodstream due to the initial onset of the interferon treatment.

The present disclosure also contemplates that the medical data management system 16 may detect both upward (increases) and downward changes (decreases) in the viral loads, and that the viral load changes may be reported through the system to the accuracy of the diagnostic test performed, such that changes of less than 0.5×10¹ copies/ml, or less than 0.4×10¹ copies/ml, or less than 0.3×10¹ copies/ml, or less than 0.2×10¹ copies/ml, or less than 0.1×10¹ copies/ml, are contemplated. For example, a viral load of one patient may change from 1×10⁷ copies/ml to around 5×10⁶ copies/ml to around 1×10⁵ copies/ml. As another example, a viral load of another patient may change from 1×10⁷ copies/ml to around 5×10⁷ copies/ml to around 1×10⁵ copies/ml. As such, both positive and negative changes in the viral loads would be monitored to the accuracy level of the diagnostic test.

With continued reference to the upper graph of FIG. 2, during a next, intermediate phase (e.g., 4-8 weeks of treatment following the initial phase described above), the viral load stays generally constant. That is, the rate of the viral load reduction (e.g., the slope of the response) has changed. In the sample response of FIG. 2, the response transitions from having a slope of substantially non-zero magnitude (the absolute value of the slope is substantially above zero) to having a slope of approximately zero. As shown in the response, during this phase, the viral load remains at approximately 1×10⁵ copies/ml. As such, the rate of the viral load reduction (or slope of the viral load response), in terms of absolute value, decreases to approximately zero. This “plateau” in viral load may reflect a transition in the patient's physiological response to the interferon—e.g., a transition from a reduction of HCV in the circulatory bloodstream to a more localized reduction of HCV in the liver.

During the final phase of the response (e.g., following the intermediate phase described above), the reduction of HCV in the bloodstream resumes. That is, the rate of the viral load reduction (e.g., the slope of the response) again changes. Here, the rate of the viral load reduction (or the slope of the viral load response), in terms of absolute value, increases from approximately zero to a substantially nonzero value. As shown in the response of FIG. 2, the viral load falls from around 1×10⁵ copies/ml to around 1×10¹ copies/ml. In the sample response of FIG. 2, the response transitions from having a slope of approximately zero to having a slope of substantially non-zero magnitude (the absolute value of the slope is substantially above zero). At the end of this phase of the treatment period, the viral load remains generally constant. Here, the treatment of the patient is considered to be successful.

The response of this patient is representative of a hypothetical patient who is successfully treated using interferon. As previously noted, however, the response curve for different patients having the same or similar treatment may differ and the example described with reference to FIG. 2 is provided as an aid to explaining a typical patient response.

For example, it is contemplated that a patient response may include one, two, three, or more than three phases such as, but not limited to, the phases described earlier. For example, in any one these phases, the viral load may decrease, increase, or trace a “plateau.” As will be explained in more detail below with reference to certain embodiments, the treatment of the patient is determined based on the patient's viral kinetics.

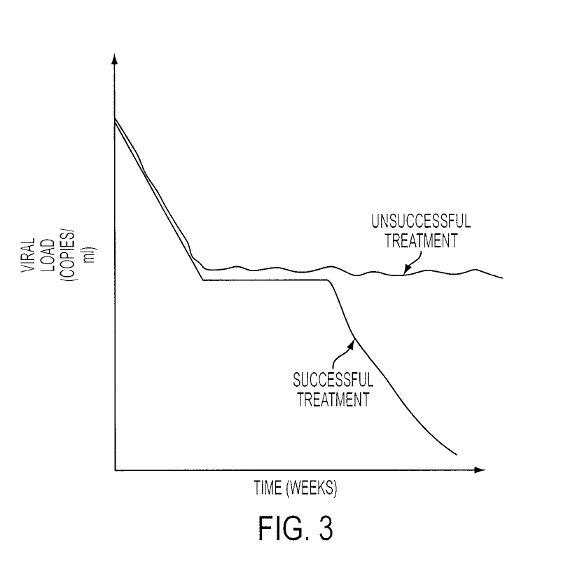

An existing standard of care typically involves administering generally a same dosage of interferon over each week, under the current standard of care of the treatment period. That is, the same quantity of interferon is supplied to the patient each week, and this dosage is applied during each week under the typical existing standard of care. For example, with reference to FIG. 3, there is a possibility that the response of the patient will take the form of a two-phase response. That is, following a fairly steep decline in the initial phase of the treatment, the viral load may tend to remain generally constant for the duration of the remaining portion of the treatment. Because the viral load remains above a nominal level of, for example, approximately 1×10⁰ copies/ml or approximately 1×10¹ copies/ml, the treatment of the patient may be considered to be unsuccessful.

Aspects of the present invention are directed towards increasing the likelihood that the treatment (such as, but not limited to, an interferon treatment) of a given patient will be successful—i.e., such that the response of the patient will (1) more closely resemble the response of FIG. 2, as described above and, conversely, (2) less closely resemble the response of FIG. 3, as also described above.

According to embodiments of the present invention, the quantity (or dosage) of interferon is varied over the treatment period. As such, the interferon can be used more effectively in reducing viral load. In more detail, according to particular embodiments, the quantity of interferon supplied to the patient at a certain time is controlled and varied based on the patient's measured (or detected) viral load. In an exemplary embodiment, viral load information is delivered to a pharmacy or medical care giver (e.g., via the system 16 to the computer 40 through the network of FIG. 1). Accordingly, a controlled quantity of interferon may be supplied to the patient. For example, in a further embodiment, which will be described in more detail below: constant dosages of a first level (e.g., a relatively low level) are supplied to the patient during an initial phase of the treatment; progressively increasing dosages (such as, but not limited to, linearly increasing dosages) are supplied to the patient during a subsequent phase of the treatment; and constant dosages of a second level (e.g., a relatively high level with respect to the first level) are supplied during a third phase of the treatment.

According to an exemplary embodiment, phases of an interferon treatment are shown in the lower graph of FIG. 2. As shown in the lower graph of FIG. 2, the treatment includes three general treatment phases. The start and end points of these treatment phases generally coincide, respectively, with the start and end points of the response phases, which were described earlier. As such, an example of the timing of the treatment phases will be described with reference to the timing of the response phases.

During a first phase of the treatment, a dosage of a first level is supplied to the patient on a periodic (e.g., weekly) basis. This first treatment phase coincides with the first response phase—i.e., the initial response phase, during which the viral load decreases fairly rapidly due to the initial onset of the interferon treatment). Here, the level of the dosage may be sized to be of a quantity suitable for killing (or at least substantially weakening) HCV that is present in the bloodstream.

During a second phase of the treatment, the dosages supplied to the patient are progressively increased (e.g., increased from the first level described above). The start point of this second phase generally coincides with the start point of the intermediate response phase—e.g., the point at which the viral load response transitions from having a slope of substantially non-zero magnitude (the absolute value of the slope is substantially above zero) to having a reduced slope, such as a slope of approximately zero (such a point will be referred to herein as an “inflection point”). In other words, while the viral load stays generally constant and/or the magnitude of the slope of the viral load response is reduced or approximately zero, the dosages are progressively (or steadily) increased. For example, according to an exemplary embodiment, as shown in FIG. 2, the dosages are controlled to be increased, so as to generally trace a ramp having a positive slope. The dosages are progressively increased during the period in which generally little change is detected in the viral load.

During a third phase of the treatment, the dosages supplied to the patient are held generally constant at a second level (e.g., a higher level than the level of the dosages at the initial phase of the treatment). Here, the second level may correspond to the peak of the ramp described above. The start point of this third phase generally coincides with the start point of the final response phase—e.g., the point at which the response transitions from having a slope of approximately zero to having a slope of substantially non-zero magnitude (the absolute value of the slope is substantially above zero). In other words, when the reduction of HCV in the bloodstream resumes (i.e., the overall slope of the viral load response has a nonzero magnitude), the dosages are held constant at (or near) the dosage level at which the reduction of HCV resumed. According to one embodiment, the dosages are held to be constant for the remainder of, for example, a 48-week treatment.

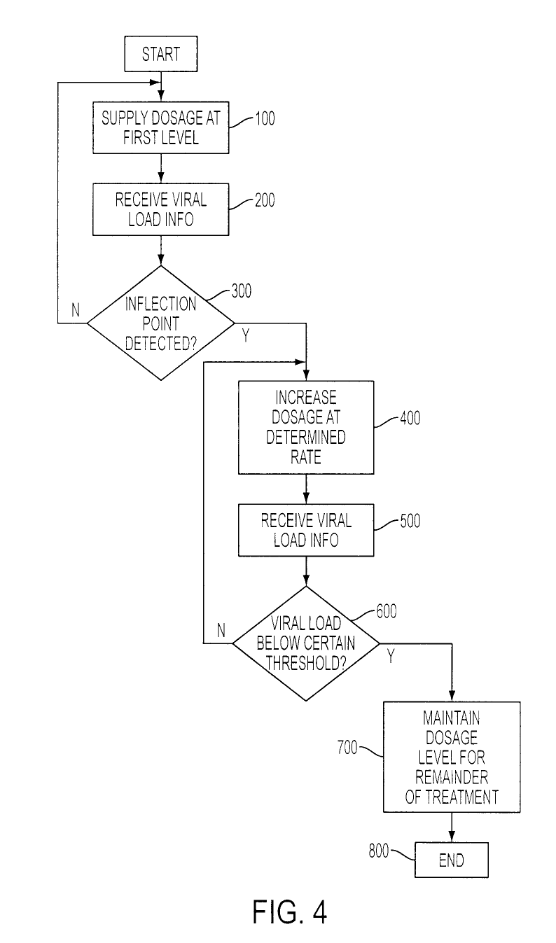

FIG. 4 shows a flow chart, according to one embodiment, of an example process of controlling dosages at a data management system 16. In box 100 of FIG. 4, the system 16 directs that a dosage of a first level be supplied to the patient. The dosage may be supplied to the patient by (or via) a pharmacy or medical care giver in communication with the system 16.

In addition, in box 200 of FIG. 4, the system 16 receives information regarding the viral load in the patient's bloodstream. According to one embodiment, the information may be provided by a medical testing facility (or clinical laboratory or the like) in communication with the system 16. Here, the patient makes periodic (e.g., weekly) visits to the facility, where his viral load is measured. However, as will be described in more detail below, in further embodiments, measurements of the viral load may be provided in environments other than medical facilities or hospitals (such as, but not limited to, home, school, or work environments where continuous, periodic and/or self testing is performed).

As explained above, the system 16 is configured to receive viral load information regarding the patient. The system 16 is further configured to determine if the slope of the viral load response (e.g., the rate of decrease of the concentration of the virus) is slowing or flattening. In other words, the system 16 is further configured to detect the presence (or lack thereof) of an inflection point in the patient's response. If the system 16 determines that the inflection point has not been reached, as represented by the “N” arm extending from box 300, then the system returns to box 100. Accordingly, a dosage of the first level is supplied to the patient at the time of the next supply. In other words, the next dosage is kept generally constant relative to the currently supplied dosage.

If the system 16 determines that an inflection point has been reached, as represented by the “Y” arm extending from box 300, then the system 16 directs that the dosage supplied to the patient be increased. As explained in more detail below, the duration (e.g., the number of weeks) over which the dosage is increased may be based on the patient's response to the increased dosages. According to one embodiment, the rate at which the dosage is increased (e.g., the slope of the ramp traced by the dosages, if the dosage is linearly increased) is based on the detected viral load at around the time of the first increase. For example, if the detected viral load is relatively low, then the rate of dosage increase will also be relatively low. If the detected viral load is relatively high, then the rate of dosage increase will also be relatively high. As such, the rate of increase is selected so as to increase the likelihood that the treatment of the patient will be successful (e.g., such that the response generally resembles the three-phase response shown in the upper graph of FIG. 2).

As explained above, the system 16 is configured to increase the dosages at a certain rate. As such, with reference to box 400 of FIG. 4, the dosage supplied to the patient is increased relative to the dosage previously supplied. Further, the system 16 is configured to continue receiving updated viral load information regarding the patient (see, for example, box 500 of FIG. 4).

With reference to box 600, the system 16 is also configured to determine if the slope of the viral load response is negative once again (e.g., the concentration of the virus in the bloodstream is, once again, declining at or above a certain rate). If the system 16 determines that the viral load is not yet declining as described above (as represented by the “N” arm extending from box 600), then the system returns to box 400. Accordingly, the dosage is again increased, and this increased dosage is supplied to the patient at the time of the next supply. In other words, the dosage is increased by an additional increment. According to a further embodiment, the determined rate by which the dosages are increased (see, for example, box 400 of FIG. 4) may itself be increased upon completion of a certain number of iterations of the “loop” beginning at box 400 and ending at box 600. Here, once a certain number of iterations has crossed a certain number, the dosages may be increased at a higher rate, e.g., to increase the likelihood that the treatment of the patient will be successful.

If the system 16 determines that the viral load is declining as described above (as represented by the “Y” arm extending from box 600), then the system 16 directs that the increases in dosage be halted (see, for example, box 700). According to one embodiment, the dosage supplied to the patient remains generally constant for the duration of the treatment period.

At the end of the treatment period, the supplying of the dosages is halted, and the treatment is ended (box 800).

A further aspect of the present invention is directed towards reducing, at least to some degree, the length of the intermediate response phase, which was described earlier. As will be described in more detail below, the likelihood that a given patient will be successfully treated can be increased because a higher drug is applied at an appropriate time or phase in time in the treatment of the patient.

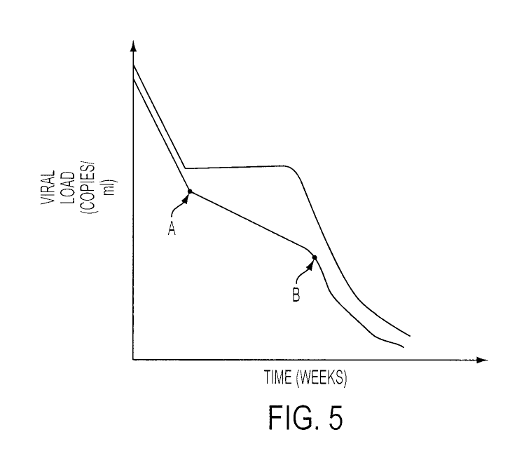

According to the embodiments described above, the dosage is ramped up when an inflection point in the viral load response occurs or is detected or sensed. As described above, the inflection point marks a point at which the viral load level becomes generally constant (e.g., the slope of the viral load response is approximately zero). However, in further embodiments, with reference to FIG. 5, the inflection point may mark a point at which the viral load level begins to fall at a slower rate (i.e., the slope of the viral load response becomes substantially smaller in magnitude) (see, for example, point ‘A’ of FIG. 5). The inflection point may mark the beginning of a phase, during which the observed or otherwise detected slope of the viral load response is smaller in magnitude than the previously observed or otherwise detected slope and dosages supplied to the patient are increased, as described above.

For example, the inflection point may mark a point at which the slope of the viral load response, in terms of absolute value, decreases by at least 25%, by at least 50%, by at least a percentage between 25% and 50%, or by at least a percentage between 50% and 100%. Here, a decrease in slope by 100% results in a response having a slope of approximately zero (e.g., a substantially horizontal line). As another example, the inflection point may mark a point at which adjacent measurements of the measured viral load response begin to be within approximately 0.5 log of each other, or within approximately 1.0 log of each other.

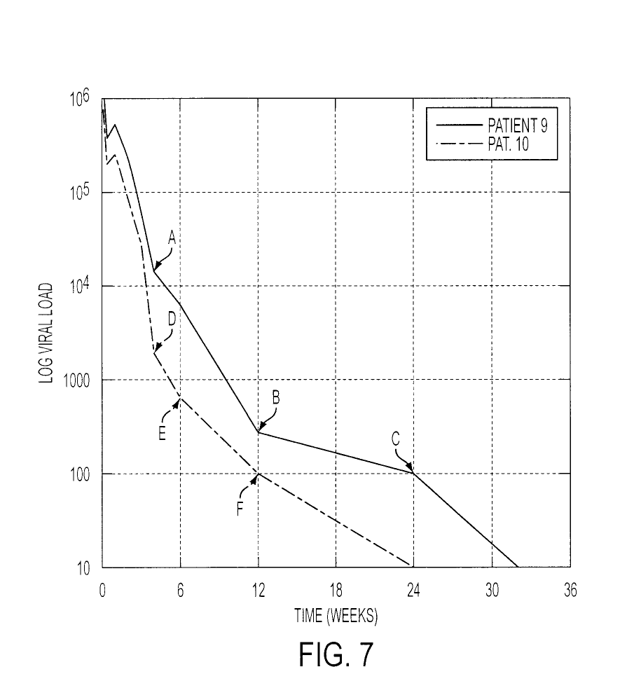

With reference to FIG. 7, two response profiles are shown, one profile corresponding to “Patient 9” and the other profile corresponding to “Patient 10.” According to one embodiment, with reference to the “Patient 9” response, the change in the slope of the response observed at point ‘A’ is sufficient to qualify as an inflection point (and, as such, may correspond to a change—e.g., an increase—in the supplied dosage). Similarly, the change in the slope of the response observed at point ‘B’ is sufficient to qualify as a second inflection point (and, as such, may correspond to another change—e.g., an increase at a higher rate—in the supplied dosage). Point ‘C’ of the “Patient 9” response corresponds to a point at which the response transitions from having a slope of generally low magnitude to having a slope of a sufficiently high magnitude (the absolute value of the slope is sufficiently high). As such, starting at the time of point ‘C’, the dosages may be held generally constant (similar to the embodiment described earlier with reference to FIG. 2). Here, according to another embodiment, if it is determined that the viral load has fallen below a certain threshold (see, for example, box 600 of FIG. 4), starting at the time of point ‘C’, the dosages are held generally constant (similar to the embodiment described earlier with reference to FIG. 2).

According to one embodiment, with reference to the “Patient 10” response, the change in the slope of the response observed at point ‘D’ is sufficient to qualify as an inflection point (and, as such, may correspond to a change—e.g., an increase—in the supplied dosage). However, the change in the slope of the response observed at point ‘E’ may not be sufficient to qualify as an inflection point (and, as such, may correspond to no change in the rate at which the dosages are changed or increased). Point ‘F’ of the “Patient 10” response also may not qualify as an inflection point. Here, according to one embodiment, if it is determined that the viral load has fallen below a certain threshold (see, for example, box 600 of FIG. 4), starting at the time of point ‘F’, the dosages are held generally constant (similar to the embodiment described earlier with reference to FIG. 2).

With reference to FIG. 5, the increase of the dosages may terminate at point ‘B’ at which the detected slope of the viral load response once again increases in magnitude.

According to embodiments of the invention, the dosages supplied may be increased at a rate such that the highest (or highest targeted) dosage supplied is a percentage of the maximum tolerated dose (MTD) of the patient. For example, the dosages supplied may be increased at a rate such that the highest (or highest targeted) dosage supplied is at least about 55%, or at least about 60%, or at least about 65%, or at least about 70%, or at least about 75%, or at least about 80%, or at least about 85%, or at least about 90%, or at least about 95%, and up to about 100%, of the maximum tolerated dose (MTD) of the patient.

According to one embodiment, the viral load is detected in a laboratory or clinic setting. That is, the patient reports to a laboratory or a clinic on a periodic basis so that tests for measuring his viral load response can be measured. According to one embodiment, the patient is tested in such an environment once per week. In other embodiments, the support device (see, for example, support device 12 of FIG. 1) includes a continuously-functioning or intermittently operated viral detector (or viral infection sensor) for continuously or intermittently detecting viral activity. For example, the viral detector may include, without limitation, a sensor that is commercially available from either Rosch Diagnostics or Beringer Manheim. In another embodiment, the viral load is detected using a personal “take home” device (e.g., a device that may be operated by the patient himself to measure his own viral load). Here, the personal device is in communication with system 16 of FIG. 1.

According to above-described embodiments, the dosage is controlled according to viral load (as detected using more traditional laboratory testing, continuous (or nearly continuous) electronic monitoring, personal “at home” testing, etc.). According to further embodiments, the dosage may be controlled according to one or more other factors or conditions in addition to viral load.

For example, in one further embodiment, the dosage may be further controlled based on the measured body temperature of the patient. Interferon is known to cause the body temperature of at least some patients to rise. As such, if a significant increase in body temperature is detected (e.g., a change of 1 degree Celsius, or a change ranging between 0.5 to 2.5 degrees Celsius), then the system 16 of FIG. 1 may respond by decreasing (e.g., at least to a slight degree) the dosage that is determined (e.g., the dosage determined according to the flowchart of FIG. 4).

Here, similar to the viral load, according to one embodiment, the patient's body temperature may be measured at a laboratory or clinical setting. In other embodiments, the body temperature may be measured by a body temperature sensor or monitor in the support device (see, for example, the support device 12 of FIG. 1). In addition, the body temperature may be measured using a personal “take home” device (e.g., a device that may be operated by the patient himself to measure his own temperature).

In the described embodiments, the body temperature may be measured concurrent with the viral load. In further embodiments, the patient's body temperature may be measured more frequently (e.g., on a daily basis, or at least twice each day) such that finer trends in the body's thermal response to the dosages can be ascertained. The application of the supplied dosages may be controlled according to such detected trends. For example, in embodiments in which the support device (see, e.g., the support device 12 of FIG. 1) includes an infusion pump (or similar infusion device), the support device may be controlled to dispense the drug (or larger rather than smaller portions of the supplied dosage) to the patient at selected times (e.g., in the morning, in the afternoon, in the evening, etc.) when the patient's body temperature is lower rather than higher. As such, the likelihood that the patient's body temperature will be elevated beyond a certain threshold temperature due to the interferon treatment can be reduced.

As another example, in a further embodiment, the dosage may be further controlled based on the patient's measured white blood cell (WBC) count. Here, interferon is known to cause the WBC count of at least some patients to fall. As such, if a significant decrease in WBC count is detected, then the system 16 of FIG. 1 may respond by decreasing (e.g., at least to a slight degree) the dosage that is determined (e.g., the dosage determined according to the flowchart of FIG. 4).

Here, similar to the viral load, according to one embodiment, the patient's WBC count may be measured at a laboratory or clinical setting. In other embodiments, the WBC count may be measured continuously or intermittently by a sensor or monitor in the support device (see, for example, the support device 12 of FIG. 1). In addition, the WBC count may be measured using a personal “take home” device (e.g., a device that may be operated by the patient himself to measure his own temperature).

Similar to the example described above, the dosage may be further controlled based on the patient's measured red blood cell (RBC) count. According to a particular embodiment, the RBC count may be of special importance if drugs such as, but not limited to, erythropoietin are required to boost the patient's RBC count.

As another example, the dosage may be adjusted according to the patient's body weight. Interferon is known to cause the body weight of at least some patients to decrease. As such, if a significant decrease in body weight is detected (e.g., such as approximately 5 pounds or approximately 2% of the patient's baseline weight), then the system 16 of FIG. 1 may respond by decreasing (e.g., at least to a slight degree) the dosage that is determined (e.g., the dosage determined according to the flowchart of FIG. 4).

Here, similar to the viral load, according to one embodiment, the patient's body weight may be measured at a laboratory or clinical setting. In other embodiments, the weight data can come from a scale (e.g., a scale designed for home use) or other suitable weight or body mass measuring device in communication with the system 16.

As another example, the dosage may be adjusted according to the amount of interferon antibodies in the patient's blood.

More generally, the dosage and/or the concentration of interferon in a supplied dosage may be controlled according to indicators (such as, but not limited to, the indicators described above). As described above, such indicators may indicate one or more aspects of the more general health of the patient. As described above, the indicators may be provided and/or measured by intermittently or continually active sensors of a support device. However, in other embodiments, the indicators may be provided by more traditional laboratory testing or “take-home” medical kits designed for use by non-professionals.

In addition, as previously described in more detail with respect to the body temperature indicator, if certain negative effects (including, but not limited to, elevated body temperature) are observed to occur more frequently at certain times with respect to other times, then the support device may be controlled to dispense the drug (or larger rather than smaller portions of the supplied dosage) to the patient at the other times.

It is contemplated that other factors may be considered by the system 16 in determining the quantity of a dosage. For example, according to one embodiment, the support device (see, for example, the support device 12 of FIG. 1) includes a mechanism (e.g., a button, a lever, or the like) operable by the patient to record any feelings or sensations of discomfort that he may feel during the course of the treatment (e.g., while he is under treatment by interferon). The system 16 may use the events logged by such a mechanism to further control the dosage. For example, in one embodiment, the logged events may be delivered to a physician or physician's assistant (e.g., by the system 16 via the network) for further evaluation. Based on the evaluation of the logged events, the dosage may be suitably controlled to reduce the occurrence and/or severity of the effects.

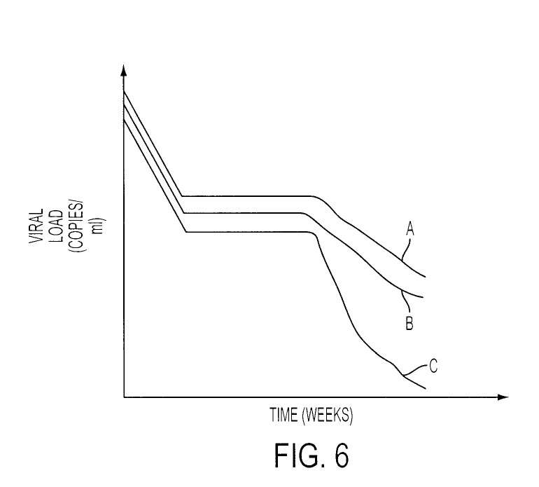

As described with respect to disclosed embodiments, dosages are controlled according to indicators such as changes in viral load levels, actual levels of the viral loads, body temperature, and like. According to a further embodiment, dosages are also controlled, at least to some extent, using experimental or clinical data. For example, with reference to FIG. 6, three response profiles are shown. Each response profile corresponds to a different dosage profile, and each dosage profile includes the following parameters: a certain dosage supplied at an initial phase of the treatment; a certain rate of dosage increase applied over an intermediate phase of the treatment; and a certain dosage supplied at a final phase of the treatment. According to the current embodiment, the system 16 analyzes such profiles and selects the dosage profile that, according to the response profiles, is most effective (or provides a desired level of effectiveness). That is, the system selects the dosage profile that facilitates the lowest end-of-treatment viral load (or otherwise provides a desired effectiveness). As such, a treatment can be based on a dosage profile that has been clinically proven to provide certain results. For example, with reference to FIG. 6, the system 16 chooses the dosage profile that corresponds to the response profile ‘C’ (over the dosage profiles that respectively correspond to the response profiles ‘A’ and ‘B’). In FIG. 6, the response profiles ‘A’ and ‘B’ are representative of patients who are not successfully cured (e.g., under an existing interferon-based treatment). In contrast, controlling dosages according to the dosage profile corresponding to the response profile ‘C’ can increase the likelihood that a patient will be successfully cured under treatments.

In a further embodiment, the dosage can be further controlled to address abnormal or undesired responses or responses substantially different from previous results. For example, similar to the intermediate treatment phase shown in the lower graph of FIG. 2 (where the dosages are increased at a certain rate), the dosages can be serially adjusted according to two or more different rates of increase (or decrease). For example, to address a detected bodily resistance to already increased dosages, the dosages may be further increased according to a second rate, which is greater than the first rate, and possibly additional progressively increasing rates. Here, the increases of the dosages can be controlled to be in conformance with both the capability of the patient to withstand the larger dosages and U.S. Food and Drug Administration (FDA) regulations relating to interferon.

Although embodiments have been described with respect to pumps, embodiments of the present invention are not limited thereto. For example, the interferon may be administered orally or via manual techniques, such as, but not limited to, manual injections.

As another example, the interferon may be administered using a combination of two or more techniques. For example, with reference to the lower graph of FIG. 2, the interferon may be administered orally during the first treatment phase (during which relatively low dosages are supplied). During subsequent phases of the treatment (i.e., the intermediate phase and the final phase, as shown, for example, in the lower graph of FIG. 2), administration of the interferon is performed via a pump-based support device, as described earlier with respect to certain embodiments.

It is also contemplated that a treatment of a patient may include aspects of existing treatments and of embodiments of the present invention. For example, treatment of a patient may begin under the current standard of care, as described earlier. As previously described, the current standard of care involves supplying a dosage of a generally static size to the patient during each week of the treatment period. The patient's response to the generally static dosages is evaluated, and further action may be based on this evaluation. For example, upon detection of an inflection point in the patient's viral load response, the dosages may be controlled (e.g., increased) according to described embodiments (see, for example, the intermediate phase of the treatment profile, as shown in the lower graph of FIG. 2). As such, selected portions (e.g., phases) of a controlled treatment according to disclosed embodiments of the present invention may be used in combination with aspects of the current standard of care.

Although embodiments have been described with reference generally to interferon, it is understood that other embodiments may cover any one or more of a range of immune modulatory therapies. These therapies include, but are not limited to, interferon alpha, interferon delta, interferon omega, etc. In addition, these therapies may include, but are not limited to, interferon conjugates, which, for example, help stabilize interferon during storage and transportation of the drug.

The foregoing description of exemplary embodiments of the invention has been presented for the purposes of illustration and description. It is not intended to be exhaustive or to limit the invention to the precise form disclosed. Many modifications and variations are possible in light of the above teachings. Therefore, it is intended that the scope of the invention be limited not by this detailed description, but rather by the claims appended hereto.

What is claimed is:

1. A method of controlling a plurality of treatment dosages for Hepatitis C, the method comprising:
setting a first dosage level to cause a viral load of a subject user to reduce at a rate over time;
determining, after setting the first dosage level, if the rate of the viral load reduction changes over time by a first threshold;
setting a second dosage level in response to determining that the rate of the viral load reduction changes by the first threshold;
determining, after setting the second dosage level, if the rate of the viral load reduction changes over time by a second threshold;
setting a third dosage level in response to determining that the rate of the viral load reduction changes by the second threshold; and
maintaining the second dosage level in response to determining that the rate of the viral load reduction has not changed by the second threshold.

2. The method according to claim 1, wherein determining if the rate of the viral load reduction changes over time by the first threshold comprises determining if the absolute value of the viral load reduction rate declines by at least a first threshold percentage.

3. The method according to claim 2, wherein the first threshold percentage is approximately 25 percent.

4. The method according to claim 3, wherein the first threshold percentage is approximately 50 percent.

5. The method according to claim 4, wherein the first threshold percentage is approximately 100 percent.

6. The method according to claim 2, wherein the second dosage level is higher than the first dosage level.

7. The method according to claim 1, wherein determining if the rate of the viral load reduction changes over time by the second threshold comprises determining if the viral load reduction rate increases by at least a second threshold percentage.

8. The method according to claim 7, wherein the first threshold percentage is approximately 25%.

9. The method according to claim 7, wherein the first threshold percentage is approximately 50%.

10. The method according to claim 7, wherein the first threshold percentage is approximately 100%.

11. The method according to claim 7, wherein the third dosage level is higher than the second dosage level.

12. The method according to claim 1, wherein the drug comprises interferon.

13. The method according to claim 1, wherein the viral load comprises a load of Hepatitis-C virus (HCV).

14. The method according to claim 1, wherein setting the second dosage level comprises setting the second dosage level further according to at least one of a body weight, a body temperature, and a white blood count of the subject user.

15. The method according to claim 1, wherein setting the third dosage level comprises setting the third dosage level further according to at least one of a body weight, a body temperature, and a white blood count of the subject user.

16. A system operable with at least one infusion device for managing medical data on an electronic communication network, the system comprising:
at least one electronic server connectable for communication on the communication network, the at least one electronic server being configured for:
receiving first viral load information of a subject user;
setting a first dosage of the infusion medium for infusion by the at least one infusion device, based on the first viral load information;
receiving second viral load information of the subject user indicative of a response of the subject user to the infusion of the first dosage; and
setting a second dosage of the infusion medium for infusion by the at least one infusion device, based on the second viral load information.
